(19) United States
(12) Patent Application Publication
(10) Pub. No.: US 2018/0148890 A1
(43) Pub. Date: May 31, 2018

(54) SYNTHETIC LEATHER MATERIALS AND METHODS OF MAKING AND USE THEREOF

(71) Applicant: BOARD OF REGENTS, THE UNIVERSITY OF TEXAS SYSTEM, Austin, TX (US)

(72) Inventors: R. Malcolm Brown, JR., Manor, TX (US); Mandy Hegemeyer, Hempstead, TX (US); Emilie Perez, Tasmania (AU); Chelsea Elisabeth Casper, Austin, TX (US); Sarah Pfeffer, Austin, TX (US)

(21) Appl. No.: 15/826,935

(22) Filed: Nov. 30, 2017

Related U.S. Application Data
(60) Provisional application No. 62/428,081, filed on Nov. 30, 2016.

(51) Int. Cl. D06N 3/00; C12P 19/04; D06N 3/02

(52) U.S. Cl. CPC D06N 3/007; D06N 3/02; D06N 3/0086; C12P 19/04

(57) ABSTRACT

Disclosed herein are synthetic leather materials and methods of making and use thereof. The methods of making the synthetic leather materials comprise: synthesizing a piece of cellulose from a microbe, thereby forming a piece of microbial cellulose; partially drying the piece of microbial cellulose; treating the partially dried piece of microbial cellulose with a conditioning agent, thereby forming a piece of conditioned microbial cellulose; drying the piece of conditioned microbial cellulose; and treating the dried piece of conditioned microbial cellulose with a hydrophobic agent, thereby forming the synthetic leather material.

### CROSS-REFERENCE TO RELATED APPLICATIONS

This application claims the benefit of U.S. Provisional Application No. 62/428,081, filed Nov. 30, 2016, which is hereby incorporated herein by reference in its entirety.

### BACKGROUND

Cellulose is the most abundant biopolymer on earth and is produced by a variety of organisms, ranging from vascular plants to algae (Czaja W et al. Cellulose, 2004, 11, 403-411). Certain bacterial strains can also produce cellulose, and each bacterial strain will create different characteristics for the cellulose material (Czaja W et al. Cellulose, 2004, 11, 403-411). Herein, the Komagataeibacter hansenii ATCC 53582 strain NQ-5 was used to grow cellulose under static culture conditions (Czaja W et al. Cellulose, 2004, 11, 403-411).

The global trade value of leather and leather products in 2010 was estimated to be $100 billion USD (United Nations Industrial Development Organization, “Future Trends in the World Leather and Leather Products Industry and Trade,” 2010). While there are many leather substitutes available on the market today, a common concern for these pseudo-leather products is their durability and aesthetics. Many of these pseudo-leather products degrade easily. The compositions and methods discussed herein address these and other needs.

### SUMMARY

In accordance with the purposes of the disclosed compositions and methods, as embodied and broadly described herein, the disclosed subject matter relates to synthetic leather materials and methods of making and use thereof.

Additional advantages of the disclosed compositions and methods will be set forth in part in the description which follows, and in part will be obvious from the description. The advantages of the disclosed compositions will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying figures, which are incorporated in and constitute a part of this specification, illustrate several aspects of the disclosure, and together with the description, serve to explain the principles of the disclosure.

FIG. 1 is a photograph of a miniature microbial cellulose prototype with a pattern engraved and dyed with red onion skin dye.

FIG. 2 is a photograph of the Timber Brown and Dark Mahogany leather dyes purchased from Tandy Leather Factory.

FIG. 3 is a photograph of the Super Shene Leather finish purchased from Tandy Leather Factory.

FIG. 4 is a photograph of the toe and heel components of the Celluleather boot prototype, the microbial cellulose components were treated with 4% and 6% PEG, adhered with an adhesive, stained 4 coats of Timber Brown Dye, and treated with 2 coats of Super Shine Sealant.

FIG. 5 is a photograph of 4% and 6% PEG treated microbial cellulose sheets, dyed with 4 coats of Dark Mahogany and treated with 2 coats of Super Shine Sealant.

FIG. 6 is a photograph of 4% PEG treated microbial cellulose sheets to be sewn behind the 4%/6% Celluleather sheet (toe and heel components), the 4% PEG treated microbial cellulose sheets were dyed with 4 coats of Timber Brown dye and treated with 2 coats of Super Shine Sealant.

FIG. 7 is a photograph of a Celluleather piece dyed with 4 coats of Dark Mahogany dye, treated with Super Shine Sealant, post laser cutter etch, and pre Super Shine Sealant second coat. The Celluleather piece comprises two layers glued together, the top layer is the one with the design laser etched therein and was treated 4% PEG, the second layer (e.g., the back side) was treated with 6% PEG.

FIG. 8 is a photograph showing the stitching on the front side of the Celluleather boot.

FIG. 9 is a photograph of a side view of the stitched front section of the Celluleather boot.

FIG. 10 is a photograph of a side view of the front and back pieces of the Celluleather boot glued together at the seams.

FIG. 11 is a photograph of a side view of the finished Celluleather boot.

FIG. 12 is a photograph of a piece of microbial cellulose treated with 4% PEG, leather dye, and leather sheen spray.

FIG. 13 is a photograph of a test strip of 4% PEG-treated microbial cellulose with leather dye submerged under water for one minute.

FIG. 14 shows a visual comparison of various test samples of microbial cellulose.

FIG. 15 shows microscopy images of various test samples of microbial cellulose.

FIG. 16 is a photograph illustrating the overlapping seam technique to make a longer sheet of microbial cellulose.

FIG. 17 is a photograph shows a 42 inches long sheet of microbial cellulose treated with 4% PEG.

FIG. 18 is a photograph of the overlapped seam (FIG. 16) after drying.

FIG. 19 is a photograph of the microbial cellulose material for a belt after applying the leather dye.

FIG. 20 is a photograph illustrating folding the dyed microbial cellulose sheet in half, length wise, to make a thicker belt and using an adhesive to secure it.

FIG. 21 is a photograph of the microbial cellulose belt after being treated with Kiwi Heavy Duty Water repellant.

FIG. 22 is a photograph of a subject wearing the microbial cellulose belt.

FIG. 23 is a photograph of the microbial cellulose belt.

FIG. 24 illustrates the process of applying multiple coats of leather dye to the microbial cellulose.

FIG. 25 shows the pieces of microbial cellulose for the moccasins after spraying the pieces with Kiwi Heavy Duty Water Repellent.

FIG. 26 illustrates the process attaching a shoe sole to the microbial cellulose using Shoe Goo.

FIG. 27 illustrates securing the shoe sole to the microbial cellulose using a needle and waxed thread.

FIG. 28 shows the stitching on the front toe area of the microbial cellulose shoe using brown embroidery thread.

FIG. 29 shows the stitching the back of the microbial cellulose shoe.

FIG. 30 is a photograph showing a side view of the finished microbial cellulose moccasins.

FIG. 31 is a photograph showing a rear view of the finished microbial cellulose moccasins.

FIG. 32 is a photograph showing a top view the finished microbial cellulose moccasins.

FIG. 33 is a photograph of a NQ4 microbial cellulose sample.

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid the reader in distinguishing the various components, features, or steps of the disclosed subject matter. The identifiers “first” and “second” are not intended to imply any particular order, amount, preference, or importance to the components or steps modified by these terms.

Disclosed herein are methods of making synthetic leather material. As used herein, a “synthetic leather material” includes a material with leather-like properties with respect to appearance, weight, texture, durability, weather resistance, hydrophobicity, flammability, or a combination thereof, but which has been synthesized (e.g., does not comprise the tanned hide of an animal). A material can be determined to have “leather-like properties” by taking a piece of test material and a piece of genuine leather, both pieces having similar dimensions, and having a population of people examine the piece of test material and the piece of genuine leather side by side in a blind examination. If a significant portion of the population cannot distinguish a substantial difference between the two pieces based on a visual and tactile review, then the piece of test material can be deemed to have “leather-like properties” (e.g., the material is a synthetic leather material).

The methods of making the synthetic leather materials can comprise synthesizing a piece of cellulose from a microbe, thereby forming a piece of microbial cellulose. The piece of microbial cellulose can be of any shape. In some examples, the piece of microbial cellulose can have a rectangular shape. The dimensions of the piece of microbial cellulose can be varied according to the intended use. The piece of microbial cellulose can, for example, have a length, a width, and a thickness, and wherein the length is from 1 inch to 100 feet or more (e.g., from 1 inch to 6 inches, from 1 inch to 1 foot, from 1 inch to 5 feet, from 1 inch to 10 feet, from 1 inch to 25 feet, from 1 inch to 50 feet, from 1 inch to 75 feet, or from 6 inches to 100 feet), the width is from 1 inch to 100 feet or more (e.g., from 1 inch to 6 inches, from 1 inch to 1 foot, from 1 inch to 5 feet, from 1 inch to 10 feet, from 1 inch to 25 feet, from 1 inch to 50 feet, from 1 inch to 75 feet, or from 6 inches to 100 feet), and the thickness is from 0.5 inches to 6 inches (e.g., from 0.5 inches to 1 inch, from 0.5 inches to 2 inches, from 0.5 inches to 3 inches, from 0.5 inches to 4 inches, from 0.5 inches to 5 inches, or from 1 inch to 6 inches). For example, the length and/or the width can be 1 inch or more (e.g., 6 inches or more, 1 foot or more, 5 feet or more, 10 feet or more, 25 feet or more, 50 feet or more, 75 feet or more, or 100 feet or more). In some examples, the thickness can be 0.5 inches or more (e.g., 1 inch or more, 1.5 inches or more, 2 inches or more, 2.5 inches or more, 3 inches or more, 3.5 inches or more, 4 inches or more, 4.5 inches or more, 5 inches or more, 5.5 inches or more, or 6 inches or more).

The microbe can be one or more prokaryotic organisms capable of generating cellulose, for example, Salmonella, Agrobacterium, Rhizobium, Nostoc, Scytonema, Anabaena, Acetobacter, Gluconacetobacter, or Komagataeibacter. In some examples, the microbe comprises a species of Komagataeibacter, such as Komagataeibacter hansenii. In some examples, the microbe can comprise the NQ5 strain of Komagataeibacter hansenii (ATCC 53582) and/or the NQ4 strain of Komagataeibacter hansenii.

The microbial cellulose can be synthesized according to known methods using standard culture conditions. The culture conditions can be varied, for example, to affect the dimensions and/or properties of the microbial cellulose. In some examples, the piece of microbial cellulose is synthesized under static culture conditions. The piece of microbial cellulose can be synthesized in an amount of time of 7 days or more (e.g., 8 days or more, 9 days or more, 10 days or more, 11 days or more, 12 days or more, 13 days or more, 14 days or more, 15 days or more, 16 days or more, 17 days or more, 18 days or more, 19 days or more, 20 days or more, 21 days or more, 22 days or more, 23 days or more, 24 days or more, 25 days or more, 26 days or more, 27 days or more, 28 days or more, or 29 days or more). In some examples, the piece of microbial cellulose can be synthesized in an amount of time of 30 days or less (e.g., 29 days or less, 28 days or less, 27 days or less, 26 days or less, 25 days or less, 24 days or less, 23 days or less, 22 days or less, 21 days or less, 20 days or less, 19 days or less, 18 days or less, 17 days or less, 16 days or less, 15 days or less, 14 days or less, 13 days or less, 12 days or less, 11 days or less, 10 days or less, 9 days or less, or 8 days or less). The amount of time in which the piece of microbial cellulose is synthesized can range from any of the minimum values described above to any of the maximum values described above. For example, the piece of microbial cellulose can be synthesized in an amount of time from 7 days to 30 days (e.g., from 7 days to 19 days, from 19 days to 30 days, from 7 days to 13 days, from 13 days to 19 days, from 19 days to 25 days, from 25 days to 30 days, from 7 days to 25 days, from 7 days to 24 days, from 7 days to 21 days, from 9 days to 19 days, from 11 days to 17 days, or from 13 days to 15 days).

The methods further comprise partially drying the piece of microbial cellulose. As used herein, “partially drying” indicates that less than 100% of the liquid is removed from the piece of microbial cellulose (e.g., at least some liquid remains in the partially dried piece of microbial cellulose). Partially drying the piece of microbial cellulose can, for example, comprise removing 75% or more of the liquid from the piece of microbial cellulose (e.g., 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, or 94% or more). In some examples, partially drying the piece of microbial cellulose can comprise removing 95% or less of the liquid from the piece of microbial cellulose (e.g., 94% or less, 93% or less, 92% or less, 91% or less, 90% or less, 89% or less, 88% or less, 87% or less, 86% or less, 85% or less, 84% or less, 83% or less, 82% or less, 81% or less, 80% or less, 79% or less, 78% or less, 77% or less, or 76% or less). The amount of liquid removed from the piece of microbial cellulose to partially dry the piece of microbial cellulose can range from any of the minimum values described above to any of the maximum values described above. For example, partially drying the piece of microbial cellulose can comprise removing from 75% to 95% of the liquid from the piece of microbial cellulose (e.g., from 75% to 85%, from 85% to 95%, from 75% to 90%, from 80% to 95%, or from 75% to 80%).

In some examples, partially drying the piece of microbial cellulose comprises pressing the piece of microbial cellulose to remove a portion of the liquid, for example pressing the piece of microbial cellulose in a hydraulic press. In some examples, partially drying the piece of microbial cellulose can comprise air drying, evaporation, partially drying in an herbarium press, freeze drying, or any other known method of drying as long as the drying can be controlled such that the piece of microbial cellulose is only partially dried.

The methods further comprise treating the partially dried piece of microbial cellulose with a conditioning agent, thereby forming a piece of conditioned microbial cellulose. As used herein, a “conditioning agent” is an agent that can affect the texture, plasticity, flexibility, or a combination thereof of the microbial cellulose. For example, the conditioning agent can be anything that gives the microbial cellulose a more “leather-like” texture and/or “leather-like” flexibility. In some examples, the conditioning agent can comprise polyethylene glycol (PEG), Tinopal LPW, carboxymethyl cellulose (CMC), derivatives thereof, or combinations thereof.

Treating the partially dried piece of microbial cellulose with the conditioning agent can, for example, comprise soaking the partially dried piece of microbial cellulose in a solution comprising the conditioning agent. In some examples, the partially dried piece of microbial cellulose can be soaked in the solution comprising the conditioning agent for 12 hours or more (e.g., 16 hours or more, 20 hours or more, 24 hours or more, 28 hours or more, 32 hours or more, 36 hours or more, 40 hours or more, or 44 hours or more). In some examples, the partially dried piece of microbial cellulose can be soaked in the solution comprising the conditioning agent for 48 hours or less (e.g., 44 hours or less, 40 hours or less, 36 hours or less, 32 hours or less, 28 hours or less, 24 hours or less, 20 hours or less, or 16 hours or less). The amount of time that the partially dried piece of microbial cellulose is soaked in the solution comprising the conditioning agent can range from any of the minimum values described above to any of the maximum values described above. For example, the partially dried piece of microbial cellulose can be soaked in the solution comprising the conditioning agent for from 12 hours to 48 hours (e.g., from 12 hours to 24 hours, from 24 hours to 48 hours, from 12 hours to 36 hours, from 16 hours to 32 hours, or from 20 hours to 28 hours).

In some examples, the conditioning agent comprises PEG and treating the partially dried piece of microbial cellulose with the conditioning agent can comprises soaking the partially dried piece of microbial cellulose in a solution comprising PEG. The concentration of PEG in the solution can be 0.1% or more (e.g., 0.5% or more, 1% or more, 1.5% or more, 2% or more, 2.5% or more, 3% or more, 3.5% or more, 4% or more, 4.5% or more, 5% or more, 5.5% or more, 6% or more, 6.5% or more, 7% or more, 7.5% or more, 8% or more, 8.5% or more, 9% or more, or 9.5% or more). In some examples, the concentration of PEG in the solution can be 10% or less (e.g., 9.5% or less, 9% or less, 8.5% or less, 8% or less, 7.5% or less, 7% or less, 6.5% or less, 6% or less, 5.5% or less, 5% or less, 4.5% or less, 4% or less, 3.5% or less, 3% or less, 2.5% or less, 2% or less, 1.5% or less, 1% or less, or 0.5% or less). The concentration of PEG in the solution can range from any of the minimum values described above to any of the maximum values described above. For example, the concentration of PEG in the solution can be from 0.1% to 10% (e.g., from 0.1% to 5%, from 5% to 10%, from 0.1% to 2.5%, from 2.5% to 5%, from 5% to 7.5%, from 7.5% to 10%, from 1% to 9%, from 2% to 8%, from 2% to 7%, or from 4% to 6%).

The methods further comprise drying the piece of conditioned microbial cellulose, for example by air drying, thereby forming a dried piece of conditioned microbial cellulose. In some examples, drying the piece of conditioned microbial cellulose can comprise placing the conditioned microbial cellulose on a substantially non-adherent surface and air drying the piece of conditioned microbial cellulose. As used herein, a substantially non-adherent surface is any surface that the piece of conditioned microbial cellulose does not substantially adhere to before, during, or after drying.

In some examples, the dimensions of dried piece of conditioned microbial cellulose with respect to length and/or width can be similar to those of the piece of microbial cellulose but with a decrease in the thickness upon drying. For example, the dried piece of conditioned microbial cellulose can have a thickness that is less than the thickness of the piece of microbial cellulose by a factor of 10 or more.

The methods further comprise treating the dried piece of conditioned microbial cellulose with a hydrophobic agent, thereby forming the synthetic leather material. The hydrophobic agent can, for example, comprise natural and synthetic oils and fats as well as waxes, silicones and functionalized silicones, surfactants and amphiphilic or hydrophobic polymers. In some examples, the hydrophobic agent can comprise a fluropolymer, an acrylic polymer, silicone, a silicon compound (e.g., a silane, a siloxane, etc.), a urethane polymer, derivatives thereof, or a combination thereof. Examples of commercially available products that can be used as the hydrophobic agent include, but are not limited to, Super Shene (Eco-Flo), Scotchgard Shoe Guard for Leather (3M), and Kiwi Heavy Duty Water Repellent (Kiwi). In some examples, the hydrophobic agent can comprise an amphiphilic compound that comprises a hydrophobic moiety and a hydrophilic moiety.

Treating the dried piece of conditioned microbial cellulose with the hydrophobic agent can, for example, comprise spin coating, drop-casting, zone casting, dip coating, blade coating, spraying, slot die coating, curtain coating, or combinations thereof. In some examples, treating the dried piece of conditioned microbial cellulose with the hydrophobic agent comprises spraying the hydrophobic agent onto the dried piece of conditioned microbial cellulose.

In some examples, the method further comprises dyeing the dried piece of conditioned microbial cellulose before treating with the hydrophobic agent. The dried piece of conditioned microbial cellulose can be dyed using any known method of dyeing. For example, the dried piece of conditioned microbial cellulose can be dyed with natural dyes, such as those made using onion skins, blueberries, spinach, coffee and tea. Traditional dyes can also be used to dye the dried piece of conditioned microbial cellulose, for example by spreading the dye on the dried piece of conditioned microbial cellulose until the desired color is achieved.

Also disclosed herein are the synthetic leather materials made by any of the methods described herein. Also disclosed herein are articles of manufacture comprising the synthetic leather materials described herein.

Examples of articles of manufacture comprising the synthetic leather materials described herein include anything that leather or animal hide is used in. For example, the article of manufacture comprising the synthetic leather materials described herein can comprise a shoe, an accessory, a clothing item, a sporting good, a home good, furniture, luggage, an animal accessory, or combination thereof.

Examples of shoes include, but are not limited to, ballet slippers, blucher shoes, boat shoes, boots, boxing shoes, brogans, brogues, cleats, climbing shoes, clogs, derby shoes, golf shoes, high heels, jazz shoes, juttis, loafers, mary janes, moccasins, mojaris, monk shoes, mules, opanaks, oxford shoes, pumps, sandals, skate shoes, sling backs, slippers, and sneakers. Examples of clothing items include, but are not limited to, jackets, skirts, pants, shorts, shirts, vests, overalls, chaps, dresses, jerkins, bodices, surcoats, capes, cowls, and corsets. Examples of sporting good include, but are not limited to, a tent, a baseball glove, a softball glove, a batting glove, a boxing glove, a shin guard, a protective head gear, a punching bag, a sports pad, a golf glove, a receiver glove, a bat bag, a golf bag, an ice skate, a goal keeper glove, a hammock, a wrestling mask, a luchador mask, an archery glove, a quiver, a bracer, a range bag, a hull bag, a shell bag, or a combination thereof. Examples of animal accessories include, but are not limited to, a collar, a leash, a harness, a saddle, a whip, a muzzle, a gentle leader, a pet carrier, a rein, a headstall, a breast collar, a halter, a lead, a stirrup, a bridle, a saddle girth, a martingale, a riding crop, a training fork, a noseband, a hackamore, a surcingale, a crupper, a pair of blinders, a chamfron, a gogue, or a combination thereof. Examples of furniture and/or home goods include, but are not limited to, a pillow, a blanket, a chair, a couch, a lampshade, a bed, a head board, a foot board, a bench, a desk, a stool, an ottoman, a chaise, a table, or a combination thereof.

In some examples, the article of manufacture comprising the synthetic leather materials described herein can comprise a duffle bag, a backpack, a messenger bag, a briefcase, a tote, a purse, a diaper bag, a saddle bag, luggage, a satchel, a handbag, a clutch, a wallet, a cuff, a keychain, a belt, a hat, a bracelet, a watch, a necklace, an earring, a ring, a basket, a tassel, a headband, a glove, a muff, suspenders, a spat, a sheath, a holster, a frogger, a baldric, a scabbard, a bandolier, a pauldron, a brigadine, a cuisee, a gauntlet, a gorget, a sabaton, a lorica segmentata, a helm, a breastplate, a bracer, an armband, greaves, polyens, a folder, a folio, a book cover, a phone case, a computer case, a tool belt, a tool bag, an apron, a guitar strap, a musical instrument case, or a combination thereof.

Also disclosed herein are methods of use of the synthetic leather materials described herein. For example, the synthetic leather materials can be used in upholstery (e.g., for furniture, automobiles, etc.) In some examples, the synthetic leather materials described herein can be used as a wrapping for a handle of an object, such as the handle of an axe, a sword, a knife, a bat, a racquet, etc.

The examples below are intended to further illustrate certain aspects of the methods and compounds described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

Preparation of the Microbial Cellulose Membrane

To obtain a high concentration microbial cellulose solution for inoculation, Komagataeibacter hansenii ATCC 53582 strain NQ5 (Laboratory Stock) was grown for 4 days in test tubes containing 10 mL Schramm and Hestrin (SH) medium at 28° C. under static conditions (Schramm M and Hestrin S. J Gen Microbiol. 1954, 11, 123-9). Pellicles were harvested and placed in two 500 ml flasks containing 100 ml Schramm and Hestrin medium supplemented with 0.8% Celluclast (cellulase). The flasks were placed on a rotary shaker set at 140 rpms and cultured for 5 days or until the microbial cellulose was completely broken down. The resulting cell solution was harvested by using a centrifugation washing process whereby the cells were spun at 3300 rpm for 10 minutes, supernatant discarded, resuspended in 50 mL of buffer (5.1 g/L Sodium Phosphate and 1.15 g/L Citric Acid), spun for another 10 minutes, washed again, and finally resuspended in 20 mL of the Acetobacter buffer. The 20 mL high concentration cell solution was used to inoculate a 24″×18″ tray containing 5 L of Schramm and Hestrin medium. The cells were allowed to culture under static conditions for 14 days. The resulting pellicle was harvested, washed with a 2% solution of Alconox, autoclave sterilized and stored in a 20% solution of Ethanol.

Preparation of the Microbial Cellulose/PEG Composite

To prepare the microbial cellulose/PEG composite material, a microbial cellulose membrane was prepared as described above. Then, 75-80% of the liquid was removed from the microbial cellulose membrane using a hydraulic press and resuspended in a 4% PEG solution for at least 24 hours (polyethylene glycol, PEG, MW 400, purchased from Sigma Aldrich). The membrane composite was dried on a sheet of Teflon at 40° C. for 24-48 hours. The PEG solution treatment gave the microbial cellulose a hide-like texture and flexibility.

### Example 2

A Celluleather prototype mini-boot was constructed using two 8″×8″ sheets of microbial cellulose synthesized by Komagataeibacter hansenii ATCC 53582 strain NQ5. Both sheets had 75-80% of the liquid removed using a hydraulic press. One sheet of microbial cellulose was treated with a 4% polyethylene glycol solution and the other sheet of microbial cellulose was treated with 6% polyethylene glycol solution. An onion dye was prepared by boiling one cup of onion skins in two cups of water, and the mixture was then cooled to give a red colored dye. The PEG-treated microbial cellulose sheets were soaked in the onion dye for 24 hours to color the PEG-treated microbial cellulose sheets. After dyeing, the sheets were stuck together using an adhesive (e.g., Elmer's glue) to form one larger dyed PEG-treated microbial cellulose sheet and left on a Teflon sheet to dry at 40° C. for 24-48 hours.

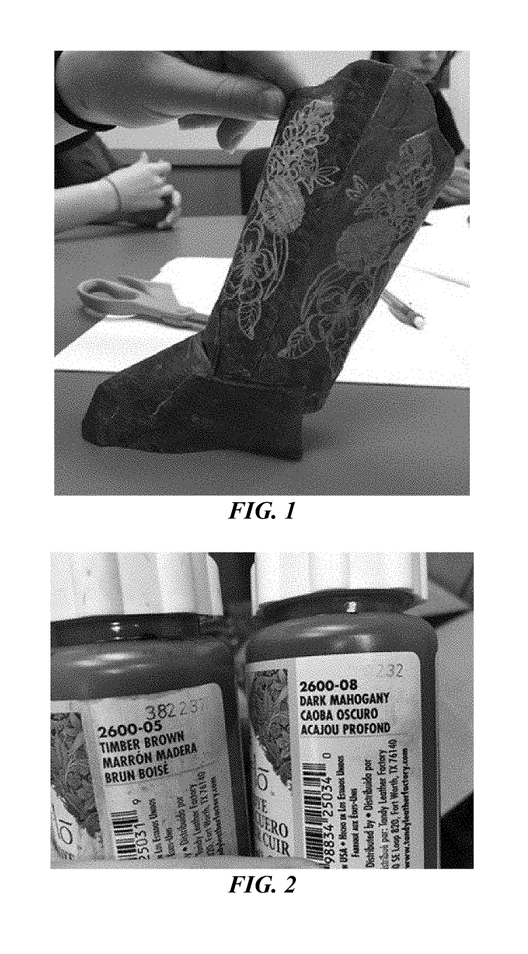

A floral design for the prototype mini-boot was created in an Illustrator program. The floral design was then etched into one of the sheets of dyed PEG-treated microbial cellulose using the leather etch setting in a laser cutter. The dyed PEG-treated microbial cellulose sheet with the finished design etched therein was measured, traced, and cut out using predetermined measurements for the mini-boot prototype. The seams of the boot were sealed together using an adhesive (e.g., super-glue). The microbial cellulose mini-boot prototype is pictured in FIG. 1. While creating this mini-boot prototype with the microbial cellulose material it was found that dyeing the PEG-treated microbial cellulose by soaking the PEG-treated microbial cellulose in a water based dye caused the microbial cellulose to lose its polyethylene glycol solution. The loss of the PEG resulted in detrimental properties for the purposes of the proposed mini-boot product, such as loss of durability and weather resistance.

### Example 3

A full sized Celluleather boot was constructed using six 14 day old 15″×22″ K. hansenii ATCC 53582 strain NQ5 microbial cellulose membranes. From 75% to 85% of the liquid was pressed out of the microbial cellulose membranes using a hydraulic press. Two of the microbial cellulose membranes were treated with a 6% polyethylene glycol solution (PEG) for 24-48 hours and four of the microbial cellulose membranes were treated with a 4% PEG solution for 24-48 hours. The PEG solution gave the microbial cellulose membranes a hide-like texture and flexibility. One 6% PEG-treated microbial cellulose sheet was sandwiched between two 4% PEG-treated microbial cellulose sheets and glued together using Elmer's Glue. The sandwiched microbial cellulose sheets were pressed to remove any trapped air and left to dry at 40° C. for at least 48 hours.

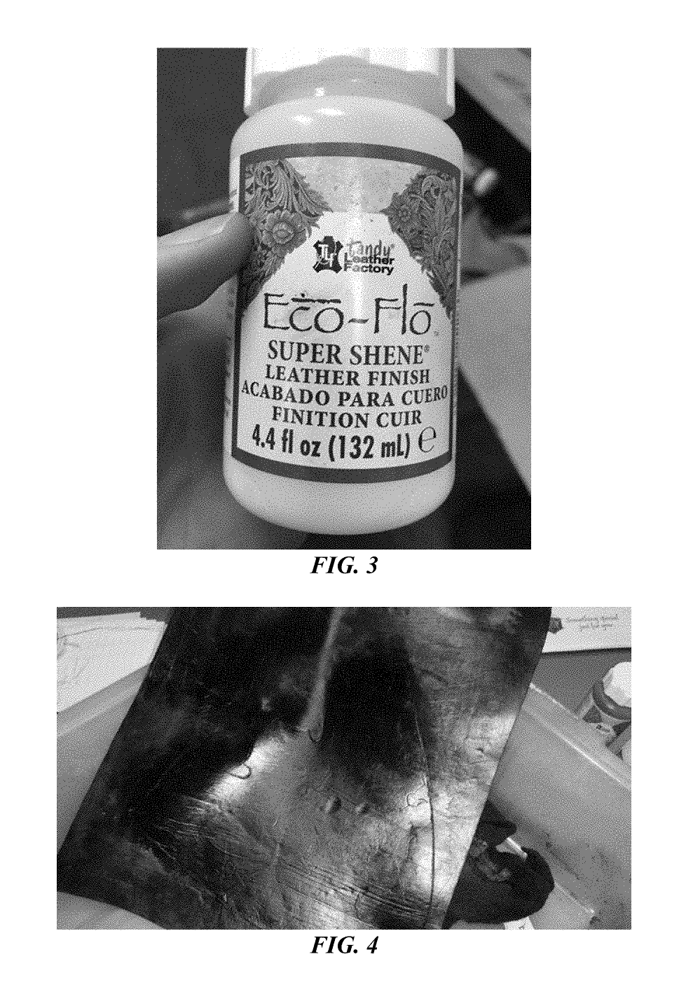

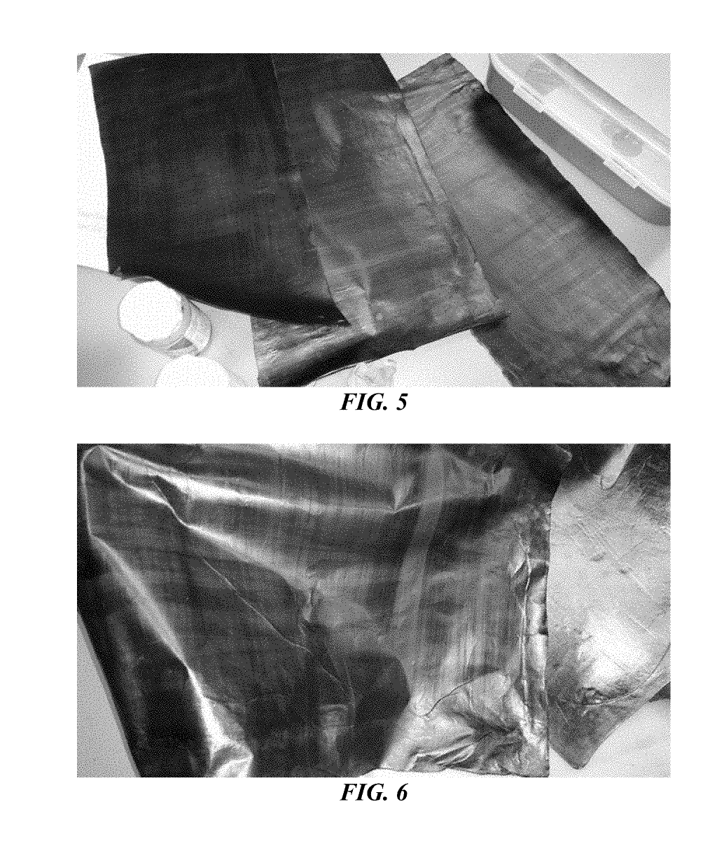

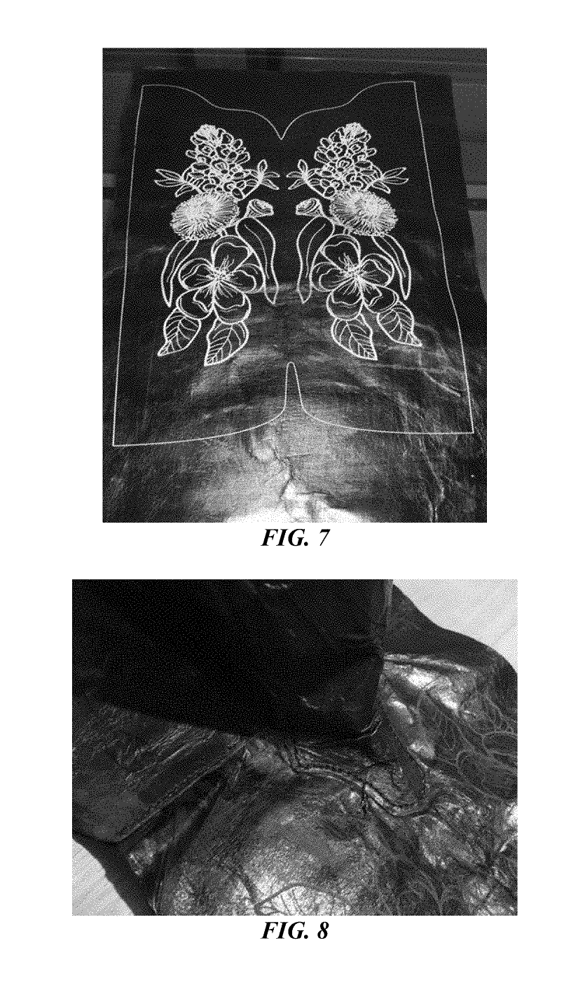

Once the sheets of PEG-treated microbial cellulose were dried, the predetermined measurements of the boot parts were traced onto the PEG-treated microbial cellulose sheets. After the boot parts were traced and measured, a cloth was used to spread dye (Tandy Leather Factory, FIG. 2) onto the PEG-treated microbial cellulose sheets in even, unidirectional strokes. The dye dried in 5-10 minutes, and the dyeing process was repeated 3-4 times using the same method, each time in a different direction. After the dyeing process was completed, a sealant (e.g., Super Shene Leather Finish, Tandy Leather Factory, FIG. 3) was applied to the dyed PEG-treated microbial cellulose (FIG. 4, FIG. 5, and FIG. 6).

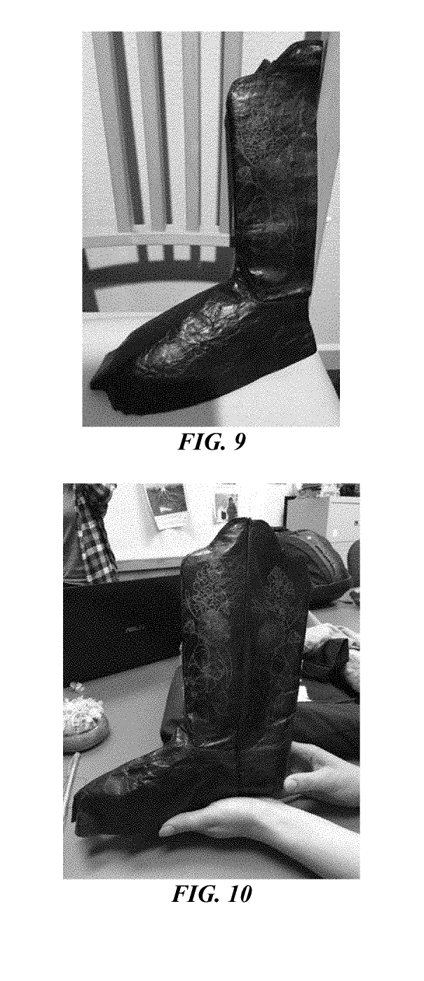

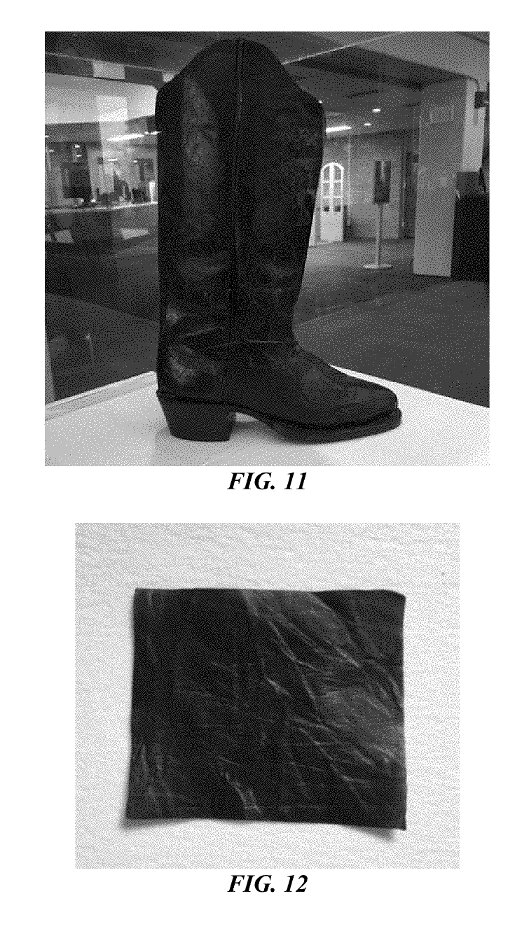

The dyed PEG-treated microbial cellulose sheets were left to dry for 48 hours, before using a laser engraving machine to etch a floral design into the dyed PEG-treated microbial cellulose sheets (FIG. 7). After etching, another layer of sealant was applied to add extra shine and seal the etched design. Each boot part was then cut out following the pre-traced design. Two thin strips (˜½-inch each) were cut out of the timber brown dyed PEG-treated microbial cellulose sheets following the top of the boot calf design and sewn to the top of both calf parts to create a seamed look. Holes were placed along the seams of the dyed PEG-treated microbial cellulose sheets using a stitching awl to allow the material to be sewn together, since the dyed PEG-treated microbial cellulose was too thick to sew with a needle by hand (FIG. 8 and FIG. 9). The seams were secured with a stitch knot (FIG. 10). After all pieces were sewn together, stretched and formed, the sole was attached. The final assembled boot made from the dyed PEG-treated cellulose material is shown in FIG. 11.

The Polyethylene Glycol treated, Komagataeibacter microbial cellulose performed well as a leather alternative.

### Example 4

Celluleather test strips were made by cutting pieces from a 14 day old 15″×22″ K. hansenii ATCC 35382 strain NQ5 microbial cellulose membrane. From 75% to 80% of the liquid was pressed out of the microbial cellulose membrane using the hydraulic press. The four small pieces of the microbial cellulose membrane were treated with a 4% PEG solution for 24-48 hours. Two of the PEG-treated microbial cellulose samples were glued together (using Elmer's glue) before they dried, after which the glued piece was dried at 40° C. on Teflon for 24 hours to make a thicker membrane (FIG. 5).

Test strips comprising the 4% PEG-treated microbial cellulose sheets were used to test the efficacy of various hydrophobic agents (e.g., water resistant sealers). Prior to the application of the hydrophobic agents, the test strips of PEG-treated microbial cellulose were dyed with a leather dye, as described above. After dyeing, each test step was dried and weighed.

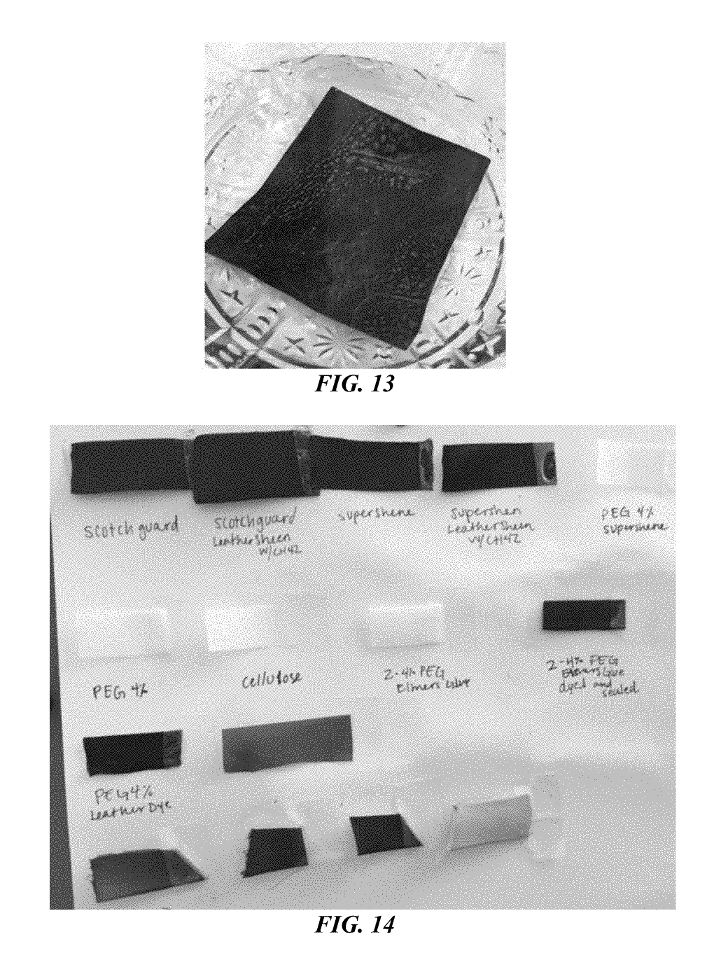

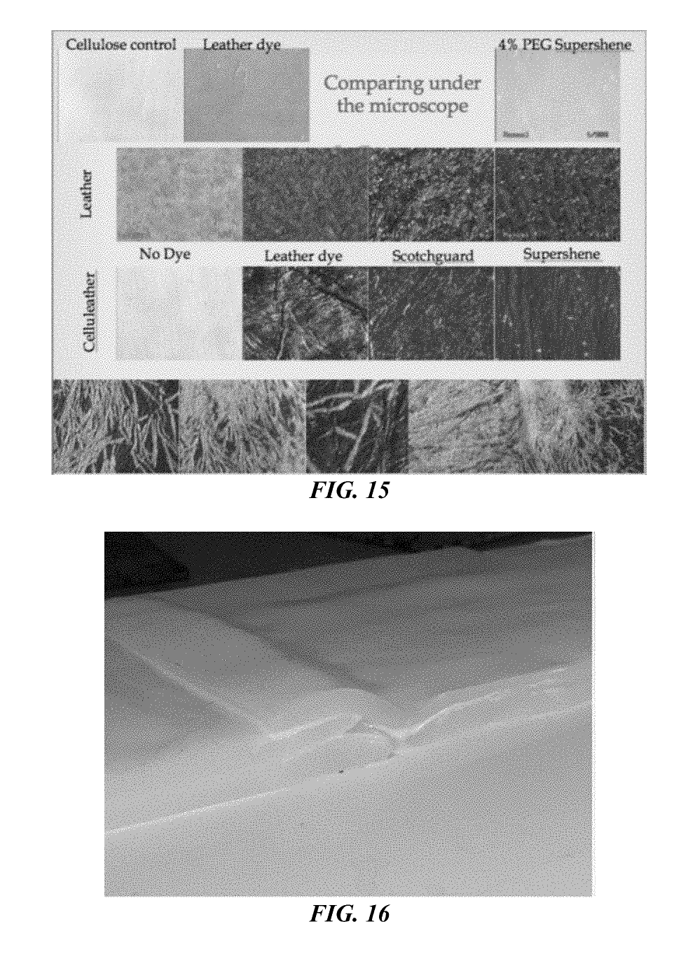

The hydrophobic agents that were tested included Scotch guard, Leather Sheen with CH42, Super Shene, and combinations thereof. An example image of a sample treated with Leather Shene with CH42 is shown in FIG. 12. To test which hydrophobic agents was most successful, the following methodology was performed: the sealant(s) was/were applied to a 4% PEG microbial cellulose sample and then the treated sample was weighed; after drying the treated sample, the dried treated sample was submerged in water for one minute (FIG. 13); after removing the sample from the water, the sample was dried and then weighed again. The weight of the sample after being submerged in water was then compared with the initial weight. Any weight loss after being submerged in water indicated that the sample lost some of its PEG, meaning that the hydrophobic agent was not successful. Photographs of the various samples are shown in FIG. 14. Microscopy images of the various samples are shown in FIG. 15. The results of the various treatments with hydrophobic agents indicated that Scotchgard was the best water repellent and made the product feel less sticky.

Microbial cellulose from Komagataeibacter was used to create a Celluleather product. The microbial cellulose is spun from spinnerets on the bacterial cells and moves across a fluid plane (e.g., a tray) leaving behind microbial cellulose membranes. Cellulose is not hydrophobic and absorbs water easily, but the Celluleather can be treated and/or sealed with a hydrophobic spray to prevent it from absorbing water. The Celluleather discussed herein is a durable, fashion-friendly, vegan alternative to leather that is just as durable and functional as traditional leather. The Celluleather absorbs color easily, is strong and durable, and less waste is generated than with other materials as the Celluleather can be grown in the amounts needed.

### Example 5

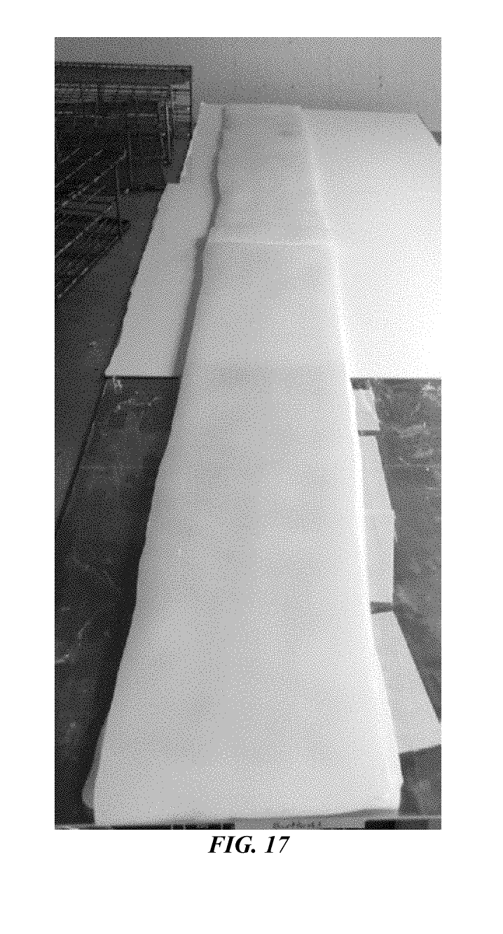

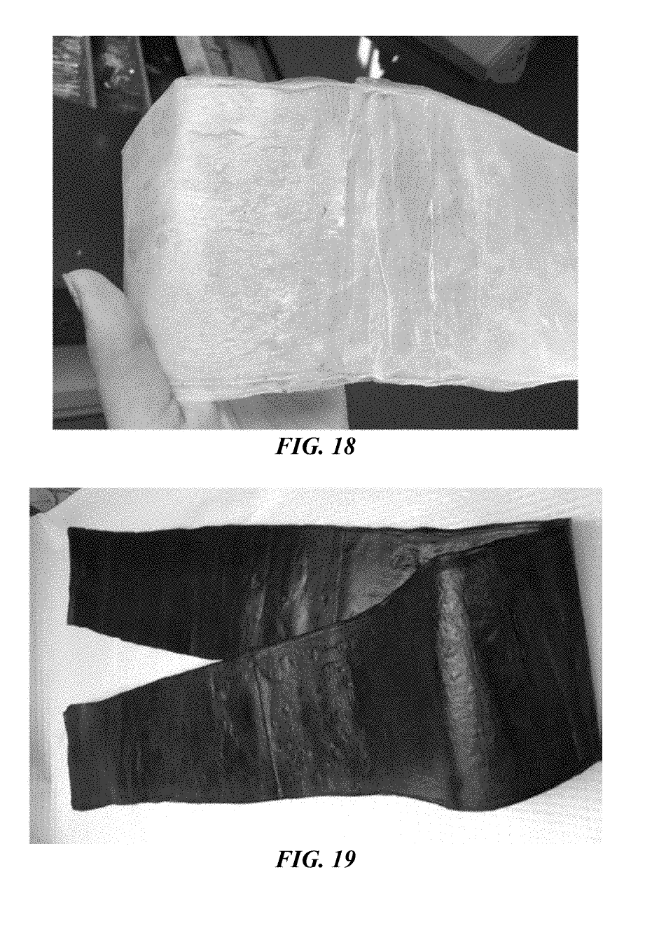

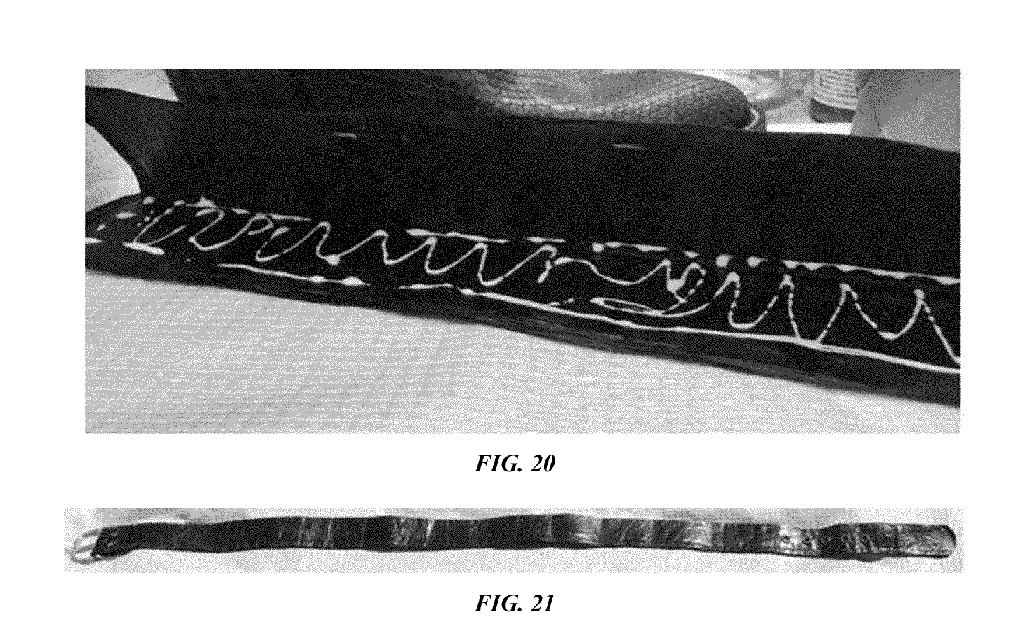

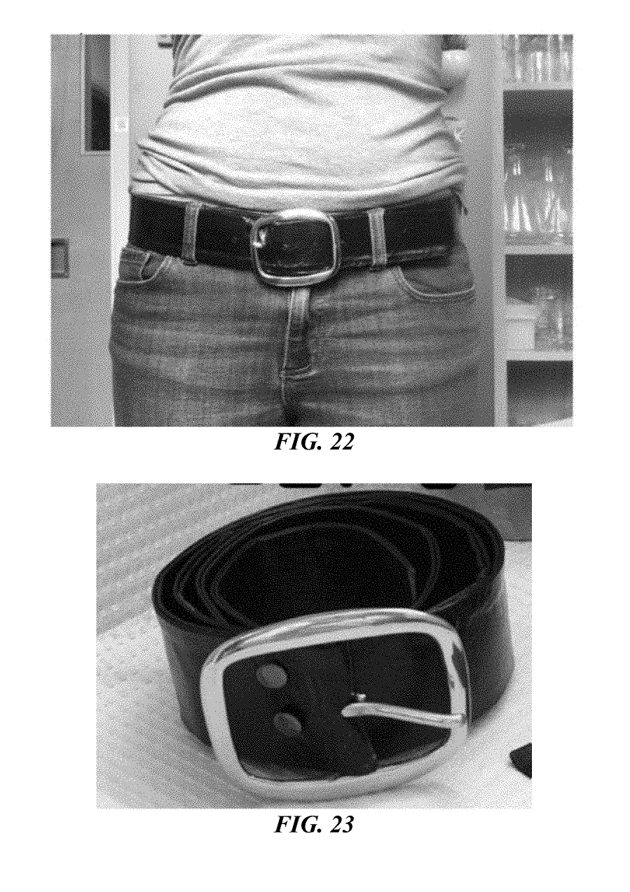

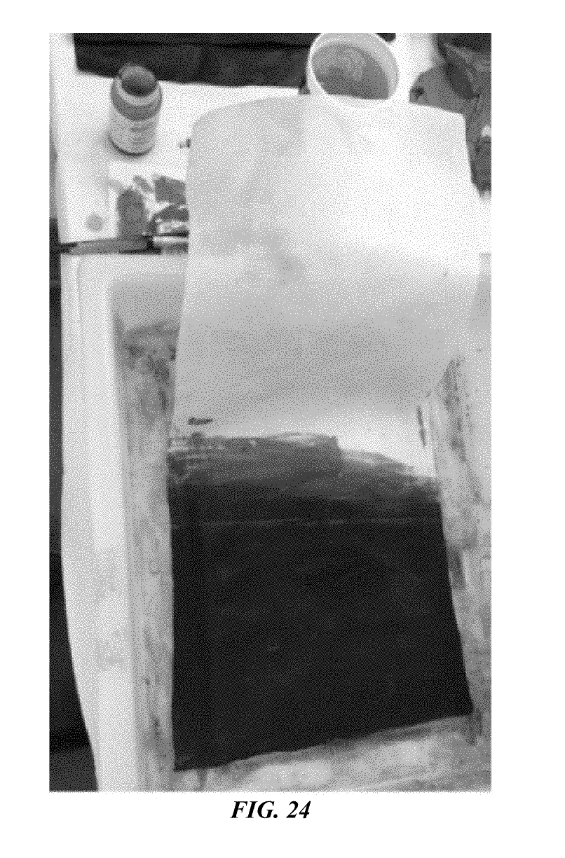

A Celluleather belt was made using sheets of a 14 day old 15″×22″ K. hansenii ATCC 53582 strain NQ5 microbial cellulose membrane that were cut into four strips that were each 5″×22″. From 75% to 80% of the liquid was pressed out of the membrane using the hydraulic press. The four strips of microbial cellulose were treated with a 4% PEG solution for 24-48 hours. The PEG-treated microbial cellulose strips were glued together (e.g., using Elmer's glue) in pairs to make the microbial cellulose thicker and stronger. To make the belt 42 inches long, the pairs of doubled sheets were glued together end to end, with the strips being overlapped in the center so they would dry sealed together (FIG. 16 and FIG. 17). The 42″ long strip was left to dry at ambient conditions on Teflon sheets. The dried strip had a smooth surface and the overlapped pieces dried together making a strong bond (FIG. 18). Two coats of Timber Brown leather dye were applied to each side of the long microbial cellulose strip using a paintbrush (FIG. 19). Once the dyed strip was dry, Scotchgard (Scotchgard Shoe Guard for Leather, 2.3-Fluid Ounce) was applied to one side of the long strip. After the Scotchgard dried, the entire strip was folded in half lengthwise and secured (e.g., using Emler's glue), so that the long strip was doubled in thickness (for additional strength) with the Scotchgarded surface now exposed on both exterior facing surfaces of the long strip (FIG. 20). A half inch seam was formed by folding each edge inwards, with the seams being secured by an adhesive (e.g., super glue). The seamed material was then cut to a predetermined size and shape, a belt buckle was attached, and belt holes were added to form the microbial cellulose belt. The microbial cellulose belt was sprayed with another water resistant spray (Kiwi Heavy Duty Water Repellent) to give the microbial cellulose belt some additional protection (FIG. 21). Finally, the microbial cellulose belt was buffed with a rag to give the microbial cellulose belt a smooth finish (FIG. 22 and FIG. 23).

The Celluleather belt was successful when tested for both strength and durability. The dye from the microbial cellulose belt stayed fast (e.g., it did not discolor any clothes that it came into contact with).

### Example 6

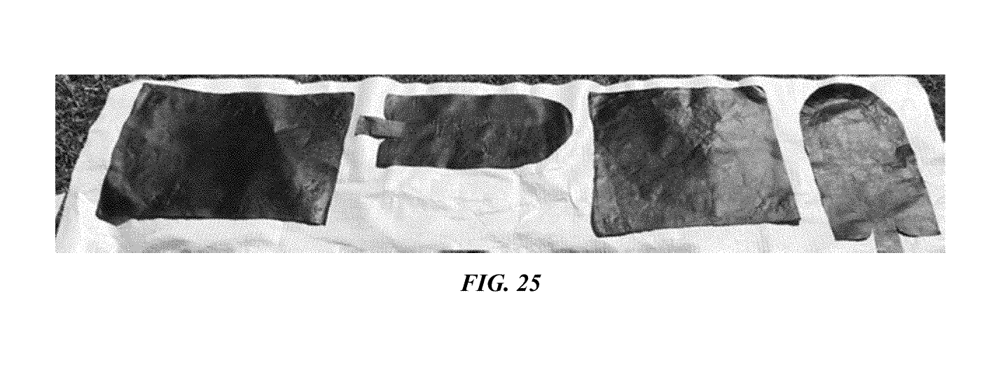

Celluleather Moccasins were constructed using 14 day old 15″×22″ K. hansenii ATCC 53582 strain NQ5 microbial cellulose membranes. The sheets of the microbial cellulose membranes were each cut into two smaller pieces (one 15″×15″ piece and one 9″×15″ piece). From 75% to 80% of the liquid was pressed out of the microbial cellulose membranes using a hydraulic press. The pressed microbial cellulose membranes were then treated with a 4% polyethylene glycol solution (PEG) for 24-48 hours, after which the PEG-treated microbial cellulose membranes were left to dry at 40° C. on Teflon sheets. The dried PEG-treated microbial cellulose membranes had a smooth surface and a hide-like texture and flexibility. Two coats of a Canyon Tan leather dye were applied to each side of the dried PEG-treated microbial cellulose membranes with a paintbrush (FIG. 24). Once the dyed PEG-treated microbial cellulose membrane was dry, Scotchgard (Scotchgard Shoe Guard for Leather, 2.3-Fluid Ounce) was applied to both sides of the dyed PEG-treated microbial cellulose membrane.

A predetermined moccasin pattern was traced onto the Scotchgard treated microbial cellulose membranes and then cut accordingly. The cut microbial cellulose sheets were then sprayed with Kiwi Heavy Duty Water Repellent (Kiwi Heavy Duty Water Repellent) on both sides to give the cut microbial cellulose pieces more protection (FIG. 25). After the Kiwi Heavy Duty Water Repellent spray was dry, the treated microbial cellulose materials were buffed with a cloth to give the material a smooth and even finish.

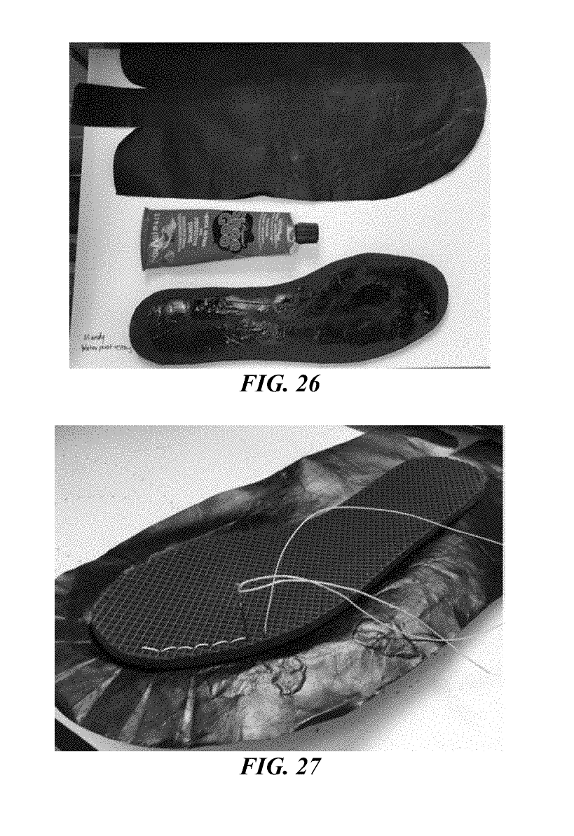

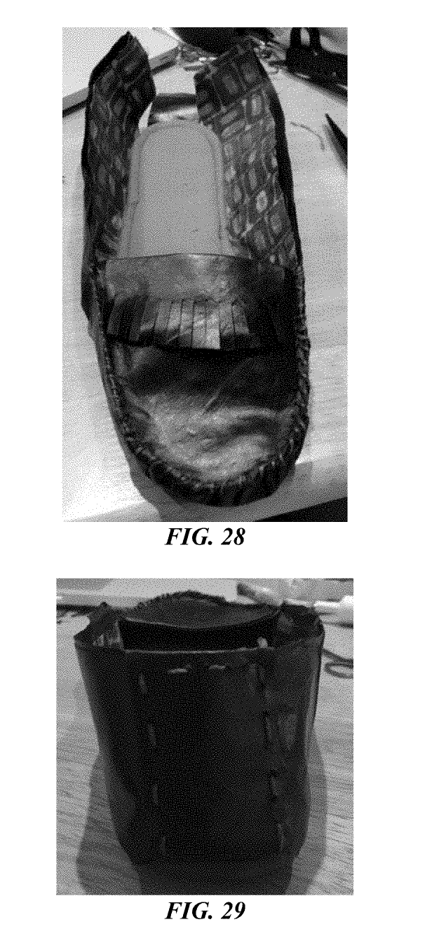

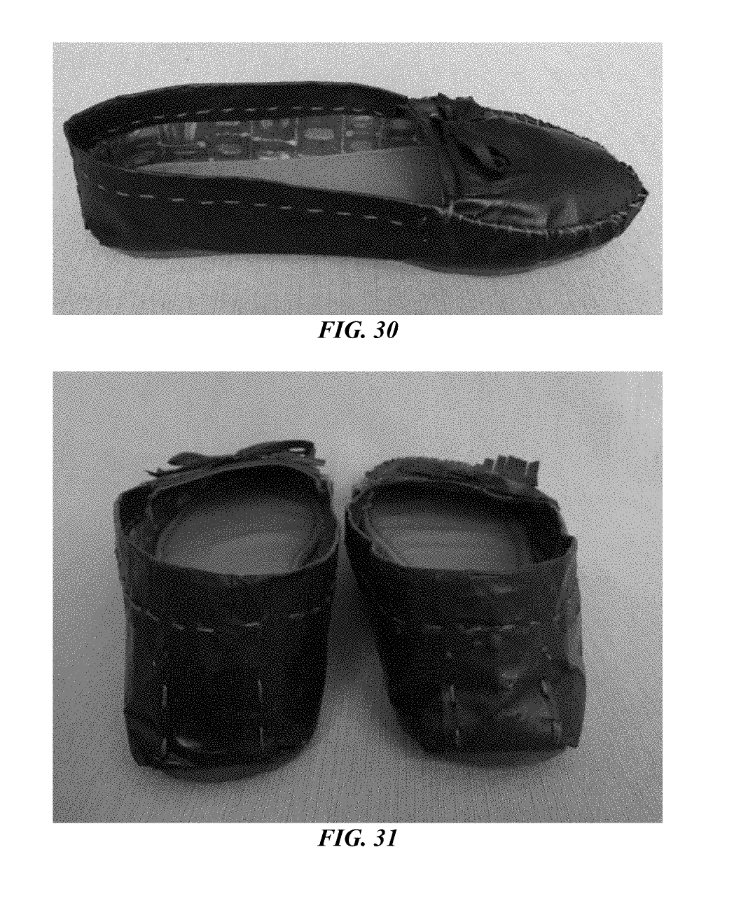

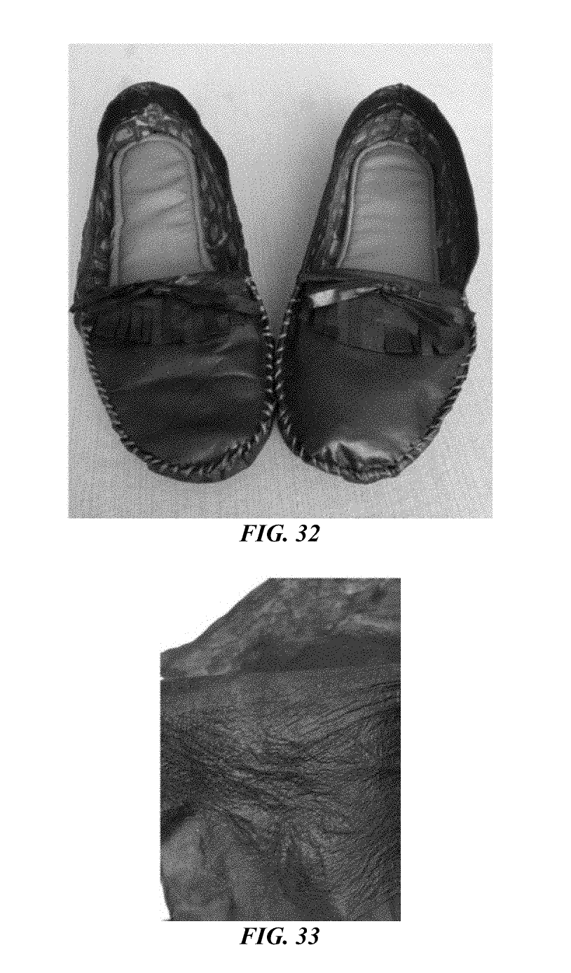

Brown soling material (SoleTech Soling Material #12P Solflex Diamond Crepe 12 Soling Material—Color Brown) was cut and sanded to a predetermined size and shape for the moccasins. The soles were then attached to the treated microbial cellulose materials with an adhesive (e.g., Shoe Goo, Black 3.7 oz), which was spread evenly on both the sole and the microbial cellulose (FIG. 26). Holes were punched through the microbial cellulose and sole and waxed thread was used to secure the sole to the microbial cellulose (FIG. 27). Another layer of microbial cellulose was then added to the shoe using an adhesive (e.g., Shoe Goo). Insoles (Dr Scholls Double Air Pillo Shoe Insole for Men & Women Memory Foam 1 Pair) were cut to a predetermined size and shape for the moccasins. The insoles were attached to the microbial cellulose with an adhesive (e.g., Liquid Stich glue). Additional pieces of microbial cellulose were used to make the top part of the shoes. The top piece of the shoe was attached to the rest of the shoe using an overcast stitch with brown embroidery thread (FIG. 28). The two side parts were similarly sewn together in the back of the shoe, after which a heel flap was attached using both an adhesive (Liquid stitch) and sewing with a leather needle (FIG. 29). Another microbial cellulose strip was attached along the edge of the shoe using an adhesive (liquid stitch) and sewing (embroidery thread). The final Celluleather Moccasins are shown in FIG. 30-FIG. 32. The Celluleather Moccasins were comfortable to wear.

### Example 7

Additional tests were conducted with a 15″×22″ K. hansenii strain NQ4 microbial cellulose membrane, which appeared be thicker than the NQ5 microbial cellulose membranes. The NQ4 microbial cellulose sheets were each cut into two smaller pieces (one 15″×15″ piece and one 9″×15″ piece). The majority of liquid was pressed out of the NQ4 microbial cellulose membranes using a hydraulic press. The pressed NQ4 microbial cellulose membranes were then treated with a 4% polyethylene glycol solution (PEG) for 24-48 hours. The PEG-treated NQ4 microbial cellulose membranes were then set out to dry on Teflon sheets at ambient conditions, which resulted in dried NQ4 microbial cellulose sheets with a smooth surface. Before the NQ4 microbial cellulose sheets were completely dry, two coats of a Canyon Tan leather dye were applied to each side of the NQ4 microbial cellulose membranes with a paintbrush. The dyed NQ4 microbial cellulose sheets were then left to dry on blotting paper in the sun. The dyed NQ4 microbial cellulose sheets dried with a different texture than the NQ5 microbial cellulose (FIG. 33 compared to FIG. 19).

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions and methods, and aspects of these compositions and methods are specifically described, other compositions and methods and combinations of various features of the compositions and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method of making a synthetic leather material, comprising:
synthesizing a piece of cellulose from a microbe, thereby forming a piece of microbial cellulose;
partially drying the piece of microbial cellulose;
treating the partially dried piece of microbial cellulose with a conditioning agent, thereby forming a piece of conditioned microbial cellulose;
drying the piece of conditioned microbial cellulose; and
treating the dried piece of conditioned microbial cellulose with a hydrophobic agent, thereby forming the synthetic leather material.

2. The method of claim 1, wherein the microbe comprises a prokaryotic microbe.

3. The method of claim 1, wherein the microbe comprises a species of Acetobacter, Gluconacetobacter, or Komagataeibacter.

4. The method of claim 1, wherein the microbe comprises Komagataeibacter hansenii.

5. The method of claim 1, wherein the microbe comprises the ATCC 53582 NQ5 strain of Komagataeibacter hansenii or the NQ4 strain of Komagataeibacter hansenii.

6. The method of claim 1, wherein the piece of microbial cellulose is synthesized under static culture conditions.

7. The method of claim 1, wherein the piece of microbial cellulose is synthesized in an amount of time from 7 days to 30 days.

8. The method of claim 1, wherein partially drying the piece of microbial cellulose comprises removing from 75% to 80% of the liquid from the piece of microbial cellulose.

9. The method of claim 1, wherein partially drying the piece of microbial cellulose comprises pressing the piece of microbial cellulose.

10. The method of claim 1, wherein treating the partially dried piece of microbial cellulose with the conditioning agent comprises soaking the partially dried piece of microbial cellulose in a solution comprising the conditioning agent.

11. The method of claim 1, wherein the conditioning agent comprises polyethylene glycol.

12. The method of claim 11, wherein treating the partially dried piece of microbial cellulose with the conditioning agent comprises soaking the partially dried piece of microbial cellulose in a solution of PEG and the concentration of PEG in the solution is from 0.1% to 10%.

13. The method of claim 1, wherein the partially dried piece of microbial cellulose is soaked in the solution comprising the conditioning agent for from 24 hours to 48 hours.

14. The method of claim 1, wherein treating the dried piece of conditioned microbial cellulose with the hydrophobic agent comprises spraying the hydrophobic agent onto the dried piece of conditioned microbial cellulose.

15. The method of claim 1, wherein the hydrophobic agent comprises an acrylic polymer, silicone, or a combination thereof.

16. The method of claim 1, wherein the method further comprises dyeing the dried piece of conditioned microbial cellulose.

17. A synthetic leather material made by the method of claim 1.

18. An article of manufacture comprising the synthetic leather material made by the method of claim 1, wherein the article comprises a shoe, an accessory, a clothing item, a sporting good, a home good, furniture, luggage, an animal accessory, or combination thereof.

19. A method of use of synthetic leather material made by the method of claim 1, the method comprising using the synthetic leather material in upholstery.

20. A method of use of synthetic leather material made by the method of claim 1, the method comprising using the synthetic leather material as a wrapping for a handle of an object.
